(19) United States
(12) Patent Application Publication
(10) Pub. No.: US 2009/0269826 A1
(43) Pub. Date: Oct. 29, 2009

(54) VACCINE AGAINST STREPTOCOCCUS AGALACTIAE INFECTION USING NATIVE OR RECOMBINANT S. AGALACTIAE GLYCERALDHEYDE-3-PHOSPHATE DEHYDROGENASE (GAPDH) AS A TARGET ANTIGEN

(75) Inventors: Paula Maria das Neves Ferreira Da Silva, Maia (PT); Maria Delfina da Conceição Tavares Gomes, Porto (PT); Patrick Trieu-Cuot, Fontenay aux Roses (FR); Manuel João Rua Vilanova, Couto (S. Miguel) (PT); Marina de Barros Nascimento Baptista, Algés (PT)

Correspondence Address: SUGHRUE MION, PLLC, 2100 PENNSYLVANIA AVENUE, N.W., SUITE 800, WASHINGTON, DC, 20037, US

(73) Assignee: Universidade Do Porto, Porto (PT)

(21) Appl. No.: 12/282,749

(22) PCT Filed: Mar. 13, 2007
(86) PCT No.: PCT/IB07/50847

§ 371 Date: Jan. 9, 2009

(30) Foreign Application Priority Data
Mar. 13, 2006 (PT) 103450

(51) Int. Cl. C12N 9/02

(52) U.S. Cl. 435/189

(57) ABSTRACT

The present invention regards a vaccine against Streptococcus agalactiae infection, a leading cause of neonatal pneumonia, sepsis and meningitis. The vaccine is composed by glyceraldheyde-3-phosphate dehydrogenase (GAPDH) protein obtained from culture supernatants of S. agalactiae cells, or by recombinant GAPDH (rGAPDH) obtained from the gene coding for the S. agalactiae GAPDH cloned and expressed in a heterologous system. The vaccine administered in a submitogenic dose of GAPDH, or rGAPDH, protects the host against S. agalactiae infection. Vaccination is used as a preventive approach and is administered by intravenous and/or intradermic and/or subcutaneous and/or mucosal route. Therefore, the invention field is in the area of the pharmaceutical industry.

### INVENTION FIELD

The present invention regards a vaccine against Streptococcus agalactiae infection, a leading cause of neonatal pneumonia, sepsis and meningitis. The vaccine is composed by glyceraldheyde-3-phosphate dehydrogenase (GAPDH) protein obtained from culture supernatants of S. agalactiae cells, or by recombinant GAPDH (rGAPDH), obtained from the gene coding for S. agalactiae GAPDH cloned and expressed in a heterologous system. The vaccine administered in a submitogenic dose of GAPDH or rGAPDH, protects the host against S. agalactiae infection. Vaccination is used as a preventive approach and is administered by intravenous and/or intradermic and/or subcutaneous and/or mucosal route. Therefore, the invention field is in the area of the pharmaceutical industry.

### Invention Early Developments

The biochemical characterization, enzymatic activity, and surface localization of Streptococcus agalactiae glyceraldheyde-3-phosphate dehydrogenase (GAPDH) protein has been described (1). However, the immunobiological effects of this protein in the host, or its use as a target antigen in a vaccine against this pathogen, have not been described.

The vaccines described against S. agalactiae are based on a capsular poly-saccharide (reviewed in 2 and 3) conjugated with tetanus toxoid (4) or with the N-terminal region of the epsilon antigen or with fragments of alpha or beta antigens from C proteins (5, 6). However, these vaccines are restricted to particular serotypes and confer protection only against these serotypes. It is known that at least five serotypes of S. agalactiae could induce meningitis in neonates. Therefore, a vaccine able to induce protection against all five serotype will be advantageous. The GAPDH protein being a ubiquitous protein is present in all serotypes.

Therefore, there are currently no vaccines capable to confer protection against all the different S. agalactiae serotypes. The protein of the present invention, S. agalactiae GAPDH, will allow overcoming this problem.

The S. agalactiae, or Lancefield's group B streptococci (GBS), infection is a leading cause of neonatal pneumonia, sepsis and meningitis. Mortality due to neonatal GBS infection remains high (0.05-0.1%), despite antibiotic therapy and 25 to 50% of surviving infants are left with permanent neurological sequelae (including sensorineural hearing loss, mental retardation, cortical blindness and seizures). In addition to the deleterious effects in newborns, GBS is also a frequent cause of infections in pregnant women, in the elderly and in immunocompromised adults.

Although there have been dramatic declines in GBS infections since the implementation of intrapartum antibiotic prophylaxis, the increase of host resistance to the used antibiotics, as well as its questionable use in pregnant humans, highlights the need for an alternative prophylactic strategy such as the development of a therapeutic or prophylactic vaccine against GBS.

In previous studies, the present inventors have demonstrated that several pathogenic microbes produce virulence-associated immunomodulatory proteins (VIP) (6-9). In mice, immunoneutralization of VIP was shown to be an effective strategy to confer host protection against systemic infections caused by the fungus Candida albicans(10). Furthermore, preventive vaccination against systemic candidiasis was attained for the first time in primates (marmosets) through immunization with an immunomodulatory protein produced by this fungus (D. Tavares, unpublished communication). Moreover, it was reported that a racemase secreted by the protozoon Trypanosoma cruzi (12) preventively protects the host from the systemic infection caused by the parasite (Patent application PCT/IB00/02008, from the Pasteur Institute et al., submitted on Dec. 4, 2000). Recently, a vaccine against dental caries was described as being based on extra cellular proteins from the cariogenic bacterium Streptococcus sobrinus and S. mutans (11 and Portuguese patent 102907).

Glyceraldheyde-3-phosphate dehydrogenase (GAPDH) protein obtained from cultures supernatants of S. agalactiae cells, or by recombinant GAPDH (rGAPDH) obtained from the gene coding for S. agalactiae GAPDH cloned and expressed in an heterologous system have been shown to possess the following biological effects:
1. stimulation of C57BL/6 mice B-cells;
2. induction of an increase in serum IL-10 in C57BL/1 mice after intraperitoneal (i.p.) treatment;
3. are associated with the virulence of S. agalactiae;
4. increase of S. agalactiae colonization in the liver of C57BL/6 mice after i.p. treatment two days before infection therefore.

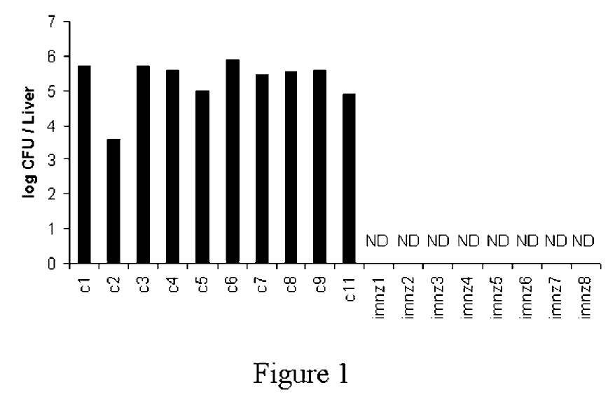

Immunoprotection assays carried out in BALB/c mice showed that immunization with GAPDH confers protection against S. agalactiae infection (FIG. 1).

### INVENTION DESCRIPTION

The aim of the present invention is the development of a vaccine against infection with S. agalactiae, an agent that causes neonatal meningitis. This vaccine comprises the glyceraldheyde-3-phosphate dehydrogenase (GAPDH) protein obtained from culture supernatants of S. agalactiae cells or by recombinant GAPDH (rGAPDH) obtained from the gene coding for S. agalactiae GAPDH cloned and expressed in a heterologous system.

This glyceraldheyde-3-phosphate dehydrogenase (GAPDH) protein, with an apparent molecular mass of 45 kDa, induces in C57BL/6 mice a B-cell stimulatory effect and induces an increase in serum IL-10 after i.p. treatment.

This protein could be considered a virulence factor for the bacterium since it facilitates the survival of the S. agalactiae in the host.

The vaccine containing the GAPDH in a submitogenic dose confers protection against systemic infection with S. agalactiae when administrated by the intraperitoneal (i.p.) route.

The vaccine against GBS infection, according to the present invention, is prepared to be administrated by intravenous and/or intradermic and/or subcutaneous and/or mucosal route to mammals, and in particular to humans.

### INVENTION DETAILED DESCRIPTION

### Purification of the Extracellular Proteins from S. agalactiae

S. agalactiae was pre-cultured in RPMI-1640 medium overnight and subsequently cultured during 48 hours in the same medium. As cultures were centrifuged at 29,000 g for 30 minutes and the supernatant cultures were filtered through a 0.22 um pore size filter and concentrated by vacuum dialysis in a Visking 100/8 FT dialysis membrane with a 30,000 Da cut-off for the collection of the extracellular proteins (EP-Sa). The absence of detectable cytosolic contaminants in EP-Sa was assessed by measuring the activity of the cytosolic isocitrate dehydrogenase using the Diagnostics Isocitrate Dehydrogenase kit. The EP-Sa was then fractionated by preparative polyacrylamide native gel electrophoresis and the fractions eluted into PBS concentrated by vacuum dialysis. All fractions were passed through a Polymixin B column to remove contaminant endotoxin and only endotoxin-free fractions, as assessed by the limulus test, were used. Protein content of the different samples was determined by the method of Lowry and the fractions were kept at −70° C. until being used.

Production and Purification of Recombinant GAPDH

The gapC gene (gbs1811; http://genolist.pasteur.fr/SagaList/) was PCR amplified in its entirety from S. agalactiae chromosomal DNA by using the primers GAP-NcoI (CCccatggTAGTTAAAGTTGG) and GAP-XhoI (CCCctcgagTTTTGCAATTTTTGC) (the restriction sites used for cloning are written in lower case). The NcoI site of the forward primer included the ATG translational start site of gapC whereas the XhoI site of the reverse primer was used to remove the stop codon. This 1021-bp long DNA fragment was digested with NcoI and XhoI and cloned into pET28a linearized with the same enzymes to produce a recombinant GAPDH containing a carboxylic histidyl tag. E. coli BL21(Y DE3) cells were transformed with the resulting recombinant plasmid (pET28aΩgapC). Following a 3-hour IPTG-induced expression of the fusion protein, the cells were harvested by centrifugation and resuspended in phosphate buffer containing 10 mM imidazole. The sample was incubated on ice for 30 min in the presence of 100 m g/ml of lysozyme and 10% Triton X-100. After sonication, the insoluble material was removed by centrifugation and the supernatant was filtered through a 0.45 μM pore size filter and applied to a His-trap column. The recombinant GAPDH was eluted with imidazole under native conditions and the eluant concentrated by vacuum dialysis and equilibrated in PBS buffer prior to endotoxin removal on a Polymixin B column as described above.

### APPLICATION EXAMPLES

### Immunoprotection Assay Using the Recombinant GAPDH (rGAPDH) as a Target Antigen

Immunization Protocol

Animal models: Female BALB/c mice aged from 8-10 weeks were bred at the Gulbenkian Institute for Science, Oeiras.

Bacteria: Streptococcus agalactiae NEM316 belongs to capsular serotype III and was isolated from a neonatal blood culture.

I) Antigens and Adjuvant.

A rGAPDH was used in a submitogenic dose. Alum (aluminium hydroxide) was used as adjuvant since it use has been licensed in humans.

II) Immunizations. Groups of 10-12 animals each were subject to the following treatment:

Female BALB/c mice were injected i.p. twice with a 3-week intervening period with 20 μg of rGAPDH plus alum (rGAPDH-immunized group) or PBS plus alum (sham-immunized control group). One month after the last immunization all the mice were i.p. infected with 5×10⁶ of S. agalactiae cells.

Challenge Infections

Fifteen days after the GBS infection, the liver was aseptically removed, homogenized in PBS and serially diluted (1:10 dilutions). Bacteria were plated onto Todd-Hewitt agar plate containing 0.001 mg/mL of colistin sulphate and 0.5 μg/mL of oxalinic acid and GBS colony-forming units (cfu) were enumerated in duplicates after 48 h of incubation at 37° C.

### Example A1

### Vaccination with rGAPDH Confers Protection Against Systemic Infection with Streptococcus agalactiae

The experiments were carried out to evaluate the effect of BALB/c immunization with submitogenic dose of rGAPDH in the protection against systemic S. agalactiae infection. As shown in FIG. 1, no detection of S. agalactiae colonization was detected in the liver of any of the mice immunized with rGAPDH, 15 days after the infection. In contrast, sham-immunized control mice present S. agalactiae colonization in the liver of all animals. Therefore, intraperitoneal immunization with rGAPDH confers protection against GBS infection.

It has been described that the main route of neonatal infection is the ascending spread of S. agalactiae into the amniotic fluid followed by the aspiration of contaminated amniotic fluid by the fetus. After gaining access to the lung, the bacteria can colonize and infect the lung, resulting in pneumonia. Subsequent transmigration of S. agalactiae across the epithelial border allows the bacteria to invade the bloodstream and eventually reach the meninges. Therefore, the mothers protected against GBS infection could prevent neonates to get infected with this bacterium.

### BIBLIOGRAPHY

1—Seifert K N, McArthur W P, Bleiweis A S, Brady L J. 2003. Can J. Microbiol. 49:350.
2—Paoletti L, Madoff L C. 2002. Semin Neonatol. 7:315.
3—Law M R, Palomaki G, Alfirevic Z, Gilbert R, Heath P, McCartney C, Reid T, Schrag S. 2005. J Med Screen 12:60.
4—US2003035805 A1 20030220 DW200316 A61K39/00 000 pp EP0866133 A2 19980923 DW199842 C12N15/70 Eng 044 pp U.S. Pat. No. 6,426,074 B1 20020730 DW200254 A61K39/09 000 pp.
5—WO1993US10506 19931102; AU19940056654 19931102; [Based on WO9410317]; ZA19930008171 19931102; FI19950001979 19950426; NO19950001629 19950428; EP19940902202 19931102; JP19940511389 19931102; NZ19930258684 19931102; HU19950001260 19931102; [CIP of] US19890408036 19890915; [Cont of] US19920968866 19921102; US19940363311 19941222; [Previous Publ. AU5665494]; [Div ex] AU19940056654 19931102; AU19980056269 19980223; [Div ex] US19940363311 19941222; US19950463288 19950605; [Div ex U.S. Pat. No. 5,648,241]; US19950470445 19950606; US19950462679 19950605; US19950466210 19950606; US19950467147 19950606; US19950469014 19950605; [Div ex AU689452]; [Previous Publ. AU5626998]; [Previous Publ. HU70981]; [Cont of] US19950469014 19950605; US19990346290 19990720; [Cont of US5968521]; KR19950701811 19950502; [Previous Publ. KR95704492]; RU19950113472 19931102.
6—Arala-Chaves M P, Ribeiro A, Vilanova M, Porto M T, Santarém, MMG, Lima M. 1988. Am J Vet Res 49: 1955.
7—Lima M, Bandeira A, Portnoi D, Ribeiro A, Arala-Chaves M P. 1992. Infect Immun 60:3571.
8—Ferreira P, Soares R, Ribeiro A, Arala-Chaves M. 1988. Scand J Immunol 27: 549.
9—Tavares D, Salvador A, Ferreira P, and Arala-Chaves M. 1993. Infect Immun 61: 1881.
10—Tavares D, Ferreira P, Vilanova M, Videira A, Arala-Chaves M. 1995. Int Immunol 7:785.
11-Dinis, M, Tavares D, Fonseca A J M M, Faria R, Ribeiro A, Cabrita A M S, Ferreira P. 2004. J Dent Res 83:354.
12—Reina-San-Martin B, Degrave W, Rougeot C, Cosson A, Chamond N, Cordeiro-Da-Silva A, Arala-Chaves M, Coutinho A, Minoprio Paola. Nature Medicine 6: 890.

1. Vaccine against Streptococcus agalactiae infection characterized by comprising the glyceraldheyde-3-phosphate dehydrogenase (GAPDH) protein obtained from culture supernatants of S. agalactiae cells, or by recombinant GAPDH (rGAPDH) obtained from the gene coding for S. agalactiae GAPDH cloned and expressed in a heterologous system.

2. Vaccine against S. agalactiae infection, in accordance with claim 1, characterized by the GAPDH protein having an apparent molecular mass of 45 kDa and being obtained from the supernatants of S. agalactiae cell cultures.

3. Vaccine against S. agalactiae infection, in accordance with claim 1, characterized by the protein obtained from the cloning of the gene (gapC) coding for the GAPDH protein of S. agalactiae and expressed in an heterologous system (like, for instance, Escherichia coli) in order to obtain the recombinant GAPDH protein.

4. Vaccine against S. agalactiae infection, in accordance with claim 3, characterized by comprising an amount of rGAPDH of 1-100 μg.

5. Vaccine against S. agalactiae infection, in accordance with claim 1 characterized by being produced for administration by intravenous and/or intradermic and/or subcutaneous and/or mucosal route.

6. Vaccine against S. agalactiae infection, in accordance with claim 1 characterized by being formulated for mammals' administration.

7. Vaccine against S. agalactiae infection, comprising the GAPDH and rGAPDH in accordance with claim 1 characterized for being used in the prevention of S. agalactiae infection.
